(19) United States
(12) Patent Application Publication
(10) Pub. No.: US 2011/0105890 A1
(43) Pub. Date: May 5, 2011

(54) MRI OPERATING METHOD

(76) Inventors: Thoralf Niendorf, Aachen (DE); Fabian Hezel, Berlin (DE)

(21) Appl. No.: 12/813,581

(22) Filed: Sep. 3, 2010

(30) Foreign Application Priority Data
Oct. 30, 2009 (EP) 09013685.4

(51) Int. Cl. A61B 5/055

(52) U.S. Cl. 600/413

(57) ABSTRACT

A magnetic-resonance imager is operated by, within the time of a R-R interval of the heart, carrying out a preparation sequence for suppressing signal contributions from the blood is carried out, in particular by a saturation sequence. At least the first refocusing pulse is generated simultaneously with a layer-selective gradient magnetic field that acts orthogonally to the layer-selective gradient magnetic field at the time of the generation of the RF excitation pulse. In addition the measuring value acquisition and image generation takes place by means of subsampling the data space and/or partially sampling the data space.

The invention relates to a method of operating a magnetic-resonance imager for spatially resolved spin resonance measurement on an object, in particular a living object, in a static magnetic field B0, where alignment of the spins of the object and a longitudinal net magnetization Mz along the magnetic field direction Z is generated, and by means of at least one radio-frequency excitation pulse in resonance, a spin-flip about a desired flip angle is generated at which a transverse magnetization component Mxy is generated or changed whose T2* relaxation is acquired for carrying out susceptibility-weighted measurements, for which purpose a metrological acquisition of a plurality of spin-echo signals from a desired volume element of the object takes place, which signals are generated after a RF excitation pulse by a sequence of radio-frequency refocusing pulses that are equidistant from one another, wherein prior to their acquisition, a susceptibility dependency is imprinted on the echo signals by an additional evolution time inserted between the radio-frequency excitation pulse and the first radio-frequency refocusing pulse for development of dephasing generated by inhomogeneities of the magnetic field, of the transverse magnetization Mxy generated by the RF excitation pulse, and wherein the location of the metrologically acquired volume element of the object is determined by gradient magnetic fields that superpose the homogenous magnetic field B0 at least temporarily, and the start and/or preparation sequences of a measurement is/are synchronized to one or more detected forms of physiological movement.

Magnetic-resonance imagers are generally known from the prior art and generally comprise coils or gradient coils for generating a plurality of, in particular, orthogonal magnetic fields, in particular in a Cartesian coordinate system, usually a coil or coil arrangement being provided to generate a strong static magnetic field B0 along a Z-direction of the selected coordinate system, for example with field strengths of several Tesla. For this, usually, superconducting coil arrangements are used.

Moreover, perpendicular and also parallel to the magnetic field direction B0 generated in this manner, further coils or coil arrangements are provided to generate magnet fields that are perpendicular to the static magnetic field and also at least one magnetic field parallel thereto, the magnetic fields being configured in particular as gradient magnet fields, i.e. magnetic fields whose magnetic field strengths change along a coordinate axis. The superposition thus ensures that the resonance frequency or precision frequency of the spin changes depending on the movable total magnetic field, spatially resolved measurements being implementable.

To be able to obtain sectional images through living objects, the measuring principle is based on the fact that the spins, in particular hydrogen spins, of the living object align themselves within the static magnetic field B0 and rotate about the magnetic field direction, thus about the Z-axis.

By means of a radio-frequency excitation pulse, which is adapted to the so-called Lamor-frequency of the rotating spin and usually is basically programmable at least with respect to the amplitude and the envelope curve, deflection of the spins out of their equilibrium can take place in such a manner that the net magnetization Mz generated in the homogenous magnetic field B0 is deflected by the so-called flip angle so that a transverse magnetization component Mxy within the XY-plane of the selected coordinate system exists. Here, the flip angle depends substantially on the RF excitation pulse and thus is programmable in an application-specific manner.

The transverse magnetization component Mxy is temporally unstable and relaxes due to different processes with different relaxation times, the different processes overlapping one another.

The person skilled in the art knows these processes that=are designated as T1-, T2 and T2*-relaxations. Here, the T1 relaxation corresponds to the increase in time of the Mz magnetization component when the Mz magnetization component flips back again in the direction of the Z-axis, whereas the T2 relaxation is based on a dephasing of the individual spins within the XY-plane and results in a weakening of the signal that is based on the radiation of an electromagnetic wave due to the rotation of the transverse magnetization component in the XY-plane.

Furthermore, the decreasing T2-signal is superimposed by a dephasing that is given by macroscopic and microscopic magnet field inhomogeneities in the examined tissue or the examined object, which thus is based on differences in the magnetic susceptibility of the tissue. The effective relaxation rate, which includes T2-relaxation as well as susceptibility effects, is designated as T2*-relaxation. Thus, the T2*-relaxation is faster than the T2-relaxation.

Beside the programming of at least temporarily acting gradient magnetic fields for achieving spatial resolution in the examined object during the measurement preparation and/or signal readout, which is principally well-known in the art, there are different possibilities for programming so-called preparation and measurement sequences of pulses for controlling the respective coils ((gradient-) magnetic field coils and/or radio-frequency coils) to make the T1-, T2-, or T2*-relaxation times metrologically recordable in a selective manner. In this context, this is also referred to as an adequate weighting regarding T1, T2, or T2* is during measurement data acquisition.

The acquisition is carried out by collecting data in the so-called data space, also called k-space, during the switched-on state within the duration of one sequence of the so-called phase-encoding gradient prior to data acquisition and the switched-on state of a so-called frequency encoding gradient during data acquisition, to achieve, in connection with the so-called layer gradient, a 3D space resolution at the time of the RF excitation pulse. The data captured in the data space are then transformed by means of a Fourier transform into an image representation.

Susceptibility-weighted measurements are of particular advantage here because they are based on magnetic field inhomogeneities within the examined tissue and are thus very sensitive with respect to the different possible types of tissue. This type of measurement is extremely error-prone so that such measurements are often superimposed on metrological artifacts that make an evaluation of the images difficult.

The central challenges of the magnetic resonance (MR) imaging of the cardiovascular system include an unaffected reproducibility, a spatial resolution in the millimeter range, and, in particular, the absolute necessity for a very detailed geometrical map of the anatomy. In addition, a susceptibility weighted MR representation of the cardiovascular system requires imaging techniques that, with adequate effect, are able to record, illustrate or quantify very small susceptibility-related signal differences between normal and abnormal tissue types in a reliable and diagnostically evaluatable manner.

A method of operating a magnetic-resonance imager is known, for example from DE 10 2007 045 172 B3 in which a spin-echo technique, which actually disables susceptibility effects, with its miscellaneous advantages could be utilized to measure susceptibility effects by introducing an additional evolution time between RF excitation pulse and first refocusing pulse to give the susceptibility effects the opportunity for a temporal development such that the they cannot be canceled out anymore by rephasing.

Here, this known technique has the disadvantage, in particular because of the measures it uses for suppressing signal contributions from the blood, that long measuring times have to be accepted, where, for example, a preparation sequence for blood suppression lies within a preceding RR-interval of the heartbeat and the actual measuring phase lies within a subsequent RR-interval. Because of the possibility that movements can take place in the meantime, another technique with a prolonged measuring time for movement compensation was used to detect organ movements and to control the measuring data acquisition depending thereon. This known technique has proven to be very accurate; however, it was too slow for clinical applications.

It is the object of the invention to provide a method of operating a magnetic-resonance imager that allows the acquisition of susceptibility-weighted measurements without having the accompanying disadvantages of known imaging techniques, in particular the associated long measuring times. Furthermore, it is the object to achieve with such a method and the programmed sequences for controlling RF pulses and the coil arrangement, an imaging that allows the suppression of artifacts caused by blood flow.

Furthermore, with the method according to the invention it is preferably possible to put an imaging technique into practice that implements high-resolution 2D or 3D views, in particular of the heart and the cardiovascular system, with high and diagnostically utilizable image quality, thereby covering a wide range of applications. This includes in particular applications such as, for example, the examination of the endothelial function of vessels, diagnosis of stress-induced angina pectoris, mapping and quantification of the iron content of myocardial tissue and the mapping of the myocardial oxygen saturation of the blood, differentiation between arteries and veins, and detection of myocardial perfusion deficits.

This object is solved according to the invention in that within the time of a RR-interval of the heart, a preparation sequence for suppressing signal contributions from the blood is carried out, in particular by means of a saturation sequence, wherein further at least the first refocusing pulse is generated simultaneously with a layer-selective gradient magnetic field that acts orthogonally to the layer-selective gradient magnetic field at the time of the generation of the RF excitation pulse, and wherein the measuring data acquisition and image generation take place by subsampling the data space and/or partially sampling the data space, in particular in connection with a Fourier transform, preferably a half-Fourier imaging.

Instead of suppressing signal contributions from the blood by global inversion of the net magnetization and subsequent layer-selective recovery, as previously done by prior-art methods, according to the invention a method of suppression of signal contributions from the blood is used to prepare the metrological acquisition, which method can be carried out within a single RR-interval of a heart and together with a further measuring sequence.

For example, for this technique of blood saturation, the so-called saturation layer can be carried out. For a heart MRI, this form of blood saturation can take place in particular by involving the pulmonary vessels and/or the atria. Here, a sequence of RF pulses is generated, in particular in a layer-selective manner, i.e. in connection with a simultaneous gradient magnet field, by means of which pulses, on average, signal contributions inclusive blood are saturated and dephased in the saturation layer and thus, the blood flowing from the area of the saturation layer into the layer to be imaged, for example heart ventricles, does not show a net magnetization in Z-direction (Mz=0) or transverse magnetization in XY-direction (Mxy=0) and hence does not deliver signal contributions after re-excitation in the layer to be imaged, for example within the heart ventricles.

By layer-selective gradient magnetic fields, such a saturation can be generated in a layer directly before and/or behind the layer that is actually to be imaged so that only saturated blood from this direction reaches the layer to be imaged and does not generate a signal contribution therein. Such saturation can be continuously generated during the MRI imaging and can be controlled by the gradient magnetic fields in such a manner that the saturation layer travels along with the layer to be imaged.

Moreover, this type of blood saturation has the advantage that it can also be used in connection with contrast agents since these agents are also being saturated.

Since with the approach of the saturation no additional waiting time between the prepared blood suppression and the time of imaging is required, this type of blood suppression can be carried out within a single heart cycle (RR-interval of the heart), and the further measurement as well. In comparison to the technique known from DE 10 2007 045 172 B3, thus a speed advantage by a factor of 2 is achieved.

The spin-echo technique, in which with a layer-selectively acting refocusing RF pulse (for example 180°), a phase inversion of the spins is generated, is further used in such a manner that the layer-selective refocusing pulse is not, as usually the case, used in the same layer as the initial excitation pulse for generating the spin flip, but in a plane arranged orthogonally thereto, for example in phase-encoding direction by simultaneous generation of the phase-encoding gradient magnetic field together with the refocusing pulse.

The rephasing and the subsequent spin echo thus takes place only from the object areas in the superimposed area of the layer selected with the excitation and the layer selected with the rephasing, which is perpendicular thereto. This results in a volume-selective excitation that downsizes the actually excited and is viewed image section and thus avoids infoldings.

Along the exemplary phase-encoding direction, i.e. the direction of the phase-encoded gradient magnetic field, thus, in this example, a reduction of the volume of the object from which the signal contributions are coming takes place so that the missing signal contributions do not need to be phase-selectively sorted.

The number of phase-encoding steps, i.e. the number of sequential switch-on procedures of the phase-encoding gradient magnetic field with different magnetic field strengths can be reduced accordingly. Also with this measure, the measuring time can be reduced, for example to 50%, with respect to the mentioned prior art.

Basically, the rephasing can also take place in frequency encoding direction by simultaneously generating the frequency encoding gradient magnetic field together with the refocusing pulse.

Apart from that further according to the invention the metrological acquisition of a plurality of spin-echo signals takes place from a desired volume element (voxel) of the object, which signals are generated after a RF excitation pulse by a sequence of radio-frequency refocusing pulses that are equidistant from one another.

The acquisition of a plurality of spin-echo signals is well known to the person skilled in the art and is based on the fact that after a RF excitation pulse for generating a spin flip about a desired flip angle, a plurality of equidistant radio-is frequency refocusing pulses are irradiated into the object to invert a dephasing of the transverse Mxy magnetization component, thus to generate a rephasing. A complete rephasing results in an echo signal that can be frequency-selectively acquired by a simultaneously switched frequency encoding gradient magnetic field with the previously specified phase encoding.

A further acceleration by at least a further factor of two is implemented in the method according to the invention by the measured value acquisition and image generation by subsampling the data space and/or the partial sampling of the data space, in particular by means of a Fourier transform, preferably a partial or half-Fourier reconstruction.

In the case of the subsampling, the invention uses the knowledge that not all increments/phases in the phase-encoding gradient magnetic field must necessarily be physically switched to acquire data of the data space, but, for example, only every second or third or other (nth) phase value, which can be integral but also rational, is switched. This results in an n-fold subsampling of the data space and thus an n-fold reduction of the data volume.

Half-Fourier utilizes the symmetry of the data space. Here, half of the data space is omitted. The reconstruction with the half-Fourier is limited to real numbers to which the imaging can usually be limited. K-t methods scan the data space in such a manner that they omit one or more data space lines.

Infoldings caused during subsampling due to the violation of the Nyquist theorem can be compensated for by suitable is reconstruction techniques and also the omitted data can be completed by suitable reconstruction techniques. For example, due to known spatial and temporal correlation in time series of image data, the “missing” information can be added, for example by the methods of the so-called “k-t-BLAST” or “k-t-PCA.”

Reconstruction techniques such as k-t-BLAST and k-t-PCA utilize previous knowledge to avoid the infoldings. In the case of k-t-BlAST, the previous knowledge exists in the form that it is assumed that the data have a periodic, recurrent behavior.

Surprisingly, it was found that these methods can also be used for MRI measurements in which in time series, signal intensity changes occur not only in selected image regions but in the entire image, as it is the case here, so that no static signal portions or image portions exist. Thus, due to the subsampling, a speed advantage of up to a factor 8 and higher is possible.

Another speed advantage results from the half-Fourier technique that, in connection with the components of the subsampling, is used with the volume-selective excitation and the blood saturation.

Here, the invention utilizes the knowledge that due to symmetries of the data space not all usually necessary phases in the phase-encoding gradient magnetic field must be switched to acquire data of the data space, but only parts of the data space are acquired and missing data space parts are completed by extrapolation or by generating complex conjugated values of existing data. The speed advantage of the approach of acquiring partial portions of the data space by means of the half-Fourier technique can be in the range of 40%.

The combination of these techniques reduces the total measuring time to less than 10-20 seconds so that there is the possibility of completely dropping the prior-art technique regarding breathing movements known from DE 10 2007 045 172 so that its low data acquisition efficiency is avoided. Complete data acquisition can thus be performed according to the invention within a breath-holding period of approximately 10 to 20 seconds.

In an advantageous development it can also be provided that the layer-selective gradient magnetic fields, which are generated at the same time as the refocusing pulses, are arranged orthogonally to one another during at least two sequential refocusing pulses.

Thus, the first refocusing pulse can be used in one layer orthogonally to the layer selected during the excitation, and on a second refocusing pulse orthogonally to the two previously selected layers. With the continuous generation of refocusing pulses, also each of the continuously selected layers can be selected to be orthogonal. Beginning with the excitation and for the selection of the layers, the gradient magnetic field can be switched, for example in the order layer gradient, phase-encoding gradient, frequency encoding gradient, and, if necessary, periodically repeating.

Hereby, the excited region can be reduced in the third dimension. Associated with this, while maintaining the spatial resolution, is a further reduction of the data volume and a potential reduction between the distances of the refocusing pulses that involves a further reduction of the image recording time.

In another configuration of the method there is the possibility of performing a diffusion weighting by generating one bipolar gradient magnetic field that acts within a limited time prior to a refocusing pulse, or two unipolar gradient magnetic fields with the same duration around a refocusing pulse. Preferably, the generation of the bipolar gradient can take place within the evolution time that follows after the RF excitation, this time not passing unutilized within a measuring sequence.

Each of the switched gradient magnetic fields, i.e. the two with different polarity and equal duration and in direct succession, or equal polarity and duration around a refocusing pulse, cause a systematic dephasing in the first switched gradient magnetic field and an inversion of the preceding dephasing, thus a rephasing, in the second one. By movements related to diffusion effects or by stochastic movements, for example of blood, after switching the pairs of bipolar and unipolar gradients, an effective net dephasing can remain that subsequently results in a decreasing signal. Thus, also signal contributions that have not been completely eliminated by the blood suppression can be considered.

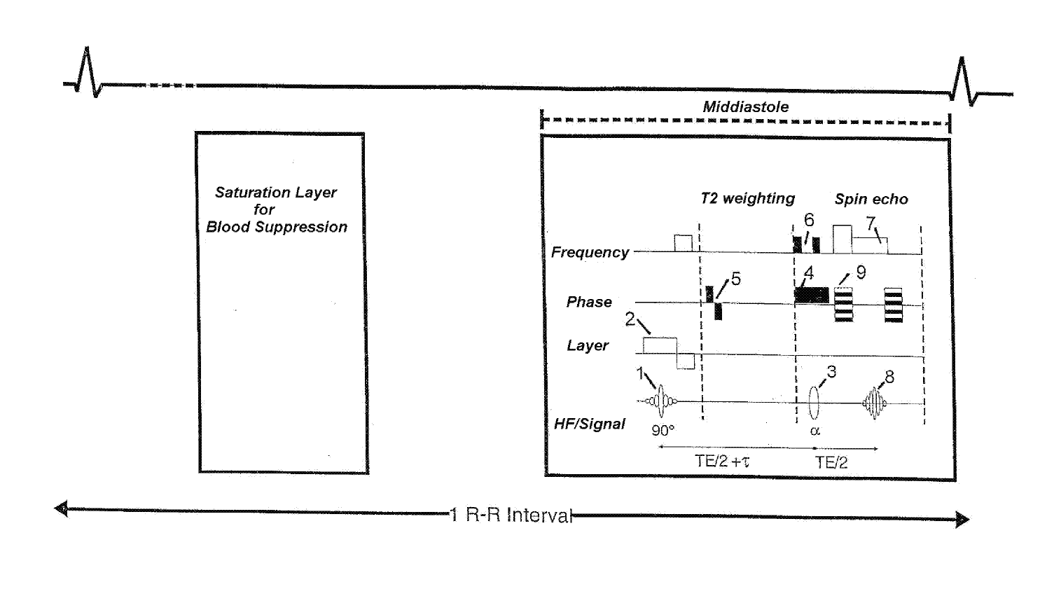

An embodiment of the invention is illustrated in FIG. 1. It shows the preparation and measuring sequences strung together for suppressing blood artifacts as well as for acquiring spin-echo signals.

The total sequence is arranged here within a RR interval of the heart. Temporally ahead of the total sequence, after the heart triggering, is a symbolically illustrated preparation sequence to avoid blood artifacts, here in particular by systematically controlling and triggering RF pulses, in particular of the layer-selective type, to achieve a disappearing magnetization in Z-direction or in the transverse plane in the blood as, for example, described above.

On the right side, in the part with the heading middiastole, as an example, a measuring sequence is illustrated that is to be carried out repeatedly for different phase encodings after an excitation and that is shown as horizontally striped representation of the phase-encoding gradient magnetic fields 9. Each of the designations “layer,” “phase,” and “frequency” indicates a switchable gradient magnetic field and all of them are arranged orthogonally to one another.

Shown here is the control within a radio-frequency coil for generating a RF excitation pulse 1, as well as the control of gradient coils for generating gradient magnetic fields to achieve a position selectivity and/or certain desired weightings during the examination of the object. Principally, the coil generating the RF pulses can also be used for acquiring the measuring signals, thus the echos, or alternatively, a separate receiving coil can be used. The excitation pulse 1 is symbolized by way of example as a 90° pulse, which turns the net magnetization from the Z-direction by 90° into the transverse plane. A layer-selective gradient magnetic field 2 acts simultaneously with the excitation pulse 1 to select the layer in the object that is to be excited by the pulse 1 to perform a spin flip. The gradient magnetic field 2 is illustrated here as bipolar with a polarity that is inverted but has only half of the duration so as to compensate a simultaneously generated dephasing.

FIG. 1 shows further that after the radio-frequency excitation pulse 1, an evolution time Tau is awaited, after which a first refocusing pulse 3 is irradiated in particular into the living object so as to repeatedly invert the dephasing of the Mxy magnetization component by a periodical sequence of further refocusing pulses and to generate the so-called spin-echo signals in this manner.

Here, the insertion of the additional evolution time Tau, which generates a total time between pulse 1 and the first refocusing pulse 3, which total time exceeds half of the equidistant temporal distance between the individual refocusing pulses 3, has the effect that prior to the first refocusing signal 3, a dephasing of the transverse magnetization component Mxy can build up that is exclusively a result of the inhomogeneities of the entire magnetic field at the location of the examination. This dephasing portion remains intact for the individual, subsequent, generated spin-echo signals and can be determined by evaluation of the spin-echo signals so that here even with the spin-echo technique, which, due to how it works, is insensitive with respect to susceptibility differences, a susceptibility-weighted imaging is made possible. Thus, a series of measurements can be recorded and evaluated for differently selected evolution times Tau.

It is essential for the invention that simultaneously with the first refocusing pulse 3, a layer-selective gradient magnetic field 4 is switched that is orthogonal to the layer-selective gradient magnetic field 2 at the time of the excitation pulse 1 so as to achieve a volume reduction since now the refocusing takes place only in the common area of both orthogonal layers. In a sequence of refocusing pulses 3 to generate successive spin echos, after each sequence step, a gradient magnetic field can be selected that is orthogonal to the previously selected gradient magnetic field. This selection sequence can be repeated. In this configuration, the gradient magnetic field 4 is selected in the phase-encoding direction for volume reduction and can be selected in frequency encoding direction for the next rephasing, etc.

The FIGURE shows also two alternatives of diffusion weighting. In one of the alternatives, at least one bipolar gradient magnetic field 5 can be generated after the excitation pulse 1 and temporally before the refocusing pulse 3. Both polarity portions have preferably the same temporal duration and the same intensity such that their active effects neutralize each other with respect to dephasing/rephasing so that diffusion processes or other movement processes, for example blood flow, which take place during the active duration, generate an effective rest dephasing that is detectable.

In the second alternative, a temporally spaced pair of unipolar gradient magnet fields 6 with equal temporal duration and equal intensity can be generated that are arranged around the refocusing pulse with respect to the time and thus also neutralize their effect with the exception of rest dephasing due to diffusion or other movement processes.

The gradient magnetic fields switched for diffusion weighting can be generated in any direction of action on individual gradient axes or as combination of a plurality of gradient directions; here, in the first alternative in the direction of the phase encoding and in the second alternative in the direction of the frequency encoding.

By means of a phase-encoding gradient magnetic field 9 that is temporarily switched prior to the measurement, the phase encoding for the subsequent measurement is defined and a spatial frequency encoding is generated with the frequency encoding gradient magnetic field 7 that is switched simultaneously with the measurement so that in this direction, the precession frequencies differ from each other.

The stripes of the phase-encoding gradient magnetic field 9 illustrate that the phase encoding is carried out multiple times in succession with a plurality of different intensities and thus phase encodings, and that then each time the frequency encoding is applied. During the frequency encoding with the frequency encoding gradient magnetic field 7, the spin echo 8 from the selected layer can be recorded with the selected phase.

The vertical bright-dark sequence illustrates that not every phase encoding, which is otherwise required, is carried out, but here symbolically only every second, or generally, every nth (n=integer or rational number) phase encoding, so that a so-called subsampling and/or partial sampling of the data space takes place resulting in a n-fold speed advantage. This can be compensated mathematically based on symmetries and correlations that are known for the data space. The evaluation of the data is then carried out according to the invention by using temporal-spatial correlations within a time series of data—known as k-t BLAST approach—and/or by means of completion of the missing data by forming complex conjugated data from the initial complex data—known as half-Fourier approach.

It is apparent that the total sequence for preparation and measurement can be carried out within only one RR interval of the heartbeat. It is apparent that the total sequence for preparatory blood suppression and measurement is independent of the T1-relaxation properties of the blood and has the advantage with respect to the prior art that it can be used without further modifications under native conditions as well as in presence of contrast agents. It is apparent that, by inserting a further delay time Tau2, which is identical to the delay time Tau and which is varied as the same and is integrated between the first and second refocusing pulse, the total sequence for preparation and measurement can be transferred without any further changes from a T2*-weighted mapping technique to a T2 mapping technique.

The combination of the techniques described here generates a speed advantage over the prior art that can exceed one order of magnitude.

1. A method of operating a magnetic-resonance imager for spatially resolved spin resonance measurement on an object, in particular a living object, arranged in a static magnetic field B0, where an alignment of the spins of the object and a longitudinal net magnetization Mz along the magnetic field direction Z is generated, wherein by means of at least one radio-frequency excitation pulse in resonance a spin-flip about a desired flip angle is generated at which a transverse magnetization component Mxy is generated or changed whose T2* relaxation is acquired for carrying out susceptibility-weighted measurements, for which purpose a metrological recording of a plurality of spin-echo signals from a desired volume element of the object takes place, which signals are generated after a RF excitation pulse by a sequence of radio-frequency refocusing pulses that are equidistant from one another, wherein prior to their acquisition a susceptibility dependency is imprinted on the echo signals by an additional evolution time inserted between the radio-frequency excitation pulse and the first radio-frequency refocusing pulse for development of the dephasing generated by inhomogeneities of the magnetic field, of the transverse magnetization Mxy generated by the RF excitation pulse, and wherein the location of the metrologically acquired volume element of the object is determined by gradient magnetic fields that superpose the homogenous magnetic field B0 at least temporarily, and the start and/or preparation sequences of a measurement is/are synchronized to one or more detected forms of physiological movement, wherein within the time of a R-R interval of the heart
a) a preparation sequence for suppressing signal contributions from the blood is carried out, in particular by a saturation sequence, and
b) at least the first refocusing pulse (3) is generated simultaneously with a layer-selective gradient magnetic field (4) that acts orthogonally to the layer-selective gradient magnetic field (2) at the time of the generation of the RF excitation pulse (1), and
c) the measuring value acquisition and image generation takes place by means of subsampling the data space and/or partially sampling the data space.

2. The method according to claim 1, wherein the layer-selective gradient magnetic fields (4), which are generated at the same time as the refocusing pulses (3), are arranged orthogonally to one another during at least two sequential refocusing pulses (3).

3. The method according to claim 1, wherein a diffusion weighting is performed by generating at least one bipolar gradient magnetic field (5) prior to a refocusing pulse or between the refocusing pulses (3), or two unipolar gradient magnetic fields (6) around a refocusing pulse (3).

4. The method according to claim 3, wherein the generation of the bipolar gradient magnetic field (5) takes place within the evolution time that follows after the RF excitation pulse (1).

5. The method according to claim 1, wherein the imaging evaluation of the data space is carried out by a half-Fourier reconstruction.
